(19) United States
(12) Patent Application Publication
(10) Pub. No.: US 2007/0280977 A1
(43) Pub. Date: Dec. 6, 2007

(54) Multiple Emulsion Excipient for Cosmetic Actives

(76) Inventors: Karla Araujo Macian, Sao Paulo (BR); Luciana Villa Nova Silva, Sao Paulo (BR); Jean Luc Gesztesi, Sao Paulo (BR); Ana Paula Pedroso de Oliveira, Sao Paulo (BR)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 11/659,649

(22) PCT Filed: Aug. 5, 2005
(86) PCT No.: PCT/BR05/00158

§ 371 Date: Jul. 27, 2007

(30) Foreign Application Priority Data
Aug. 6, 2004 (BR) PI0403269-1

(51) Int. Cl. A61K 8/06; A61K 47/00; A61Q 19/00

(52) U.S. Cl. 424/401; 514/772

(57) ABSTRACT

The present invention relates to a P/O/W-type multiple emulsion, constituted by two phases: an aqueous phase composed of an oil-in-water emulsion and an oil phase composed of a polvol-in-oil emulsion, wherein said oil phase comprises at least one lipophilic emulsifying agent, at least one lipophilic co-emulsifying agent and at least one electrolyte. This multiple emulsion is intended to be used as an excipient for actives, since it protects them from oxidation and hydrolysis, promotes delay or control of the release thereof and reduces the possible irritation caused by said actives.

This application claims the priority of Brazilian patent case No. P10403269-1 filed on Aug. 6, 2004 which is hereby incorporated by reference.

### FIELD OF THE INVENTION

The present invention relates to a P/O/W-type emulsion, which is constituted by two phases: an aqueous phase composed by an oil-in-water emulsion and an oil phase composed by a polyol-in-oil type emulsion. This multiple emulsion is intended to be used as an excipient for actives, since it protects them against oxidation and hydrolysis, promotes the delay or control of their release and reduces the irritation that may be caused by said actives.

### DESCRIPTION OF THE PRIOR ART

A multiple emulsion is a complex system that may be regarded as being emulsions made of emulsions. Multiple emulsions are formed by a dispersion of droplets that contain event smaller droplets of a phase equal or similar to the continuous outer phase. This type of emulsion has a great potential of use in systems of controlled drug release.

The types of multiple emulsions may vary according to the chemical nature of the emulsion droplets dispersed and the chemical nature of the outer phase. There are two main types of multiple emulsion:
1—W/O/W (water-in-oil-in-water), in which droplets of a W/O (water-in-oil) emulsion are dispersed in an aqueous outer phase;
2—O/W/O (oil-in-water-in-oil), in which droplets of an O/W (oil-in-water) emulsion are dispersed in an oily outer phase.

Further, there are the following variations:
1.1—W1/O/W1—the inner aqueous phase is equal to the outer aqueous phase;
1.2—W1/O/W2—the inner water phase is different from the outer aqueous phase;
2.1—O1/W/O1—the inner oil phase is equal to the outer oil phase;
2.2—O1/W/O2—the inner oil phase is different from the outer oil phase.

Multiple emulsions may be used as controlled-active-release systems for “in situ” separate incompatible raw materials of the formula and to protect the hydrophilic actives against hydrolysis and oxidation. A drug or actives dispersed in the inner droplets may be gradually released, which promotes a prolonged effect.

Some documents of the prior art deal with processes of preparing emulsions, including multiple emulsions, namely:

Document U.S. Pat. No. 5,543,135 discloses a process of preparing a water-in-oil emulsion that comprises a step of mixing an oil dispersion of droplets of a metallic oxide having primary particle size smaller than 0.2 micron with one or more emulsifying agents and an aqueous phase. Small amounts of emulsifiers are used.

Document WO 92/18227 describes multiple composition emulsions comprising a mixture of emulsifiers, one being hydrophobic and the other being hydrophilic, wherein each of the components should exhibit specific properties referring to solubility, isotropicity, among others.

Finally, document U.S. Pat. No. 6,171,600 discloses an X/O/Y type multiple emulsion containing at least one X/O phase, O being an oil and X being an oil-immiscible component. Y may be an aqueous phase or a water-in-oil type emulsion. Actives may be added to the X/O phase. Further, a process for preparing said multiple emulsion is described.

The multiple emulsion described in this latter document has drawbacks with respect to stability. From tests carried out, one has concluded that the stability of said emulsion ends in a period of 15 days, due to the breakage of the droplets. Right after said period, the phases of the emulsion separate from each other, being seen with the naked eye, thus decharacterizing the emulsion.

As can be inferred from the description of the present invention hereinafter, no teaching of the prior art proposes a multiple emulsion composed of two emulsions (forming 4 phases), in addition to the advantages referring especially to the stability of the emulsion and to the various possibilities of use of a multiple emulsion foreseen in the present invention.

### OBJECTIVES OF THE INVENTION

The present invention has the objective of providing a multiple emulsion to be used as a cosmetic or a system of controlled release of actives, wherein said multiple emulsion of the P/O/W type is constituted by two phases: an aqueous phase composed of an oil-in-water type emulsion and an oil phase composed of a polyol-in-water type emulsion, and said oil phase comprises at least one emulsifying agent and one co-emulsifier agent, and the aqueous phase comprises at least one electrolyte. This multiple emulsion may further contain several components such as vitamins, enzymes, antiperspirant actives, fragrances and other components known in the cosmetology area, including components incompatible with the outer aqueous phase.

### SUMMARY OF THE INVENTION

The invention has the objective of providing a multiple emulsion of the P/O/W type, which is constituted by two phases an aqueous phase composed by an oil-in-water type emulsion and an oil phase composed by a polyol-in-oil type emulsion that comprises at least one lipophilic emulsifying agent, at least one lipophilic co-emulsifying agent and at least one electrolyte.

Further, the present invention relates to a cosmetic product that comprises the above-described multiple emulsion.

### DETAILED DESCRIPTION OF THE INVENTION

A multiple emulsion is a system of controlled release of actives, obtained by an encapsulating process that consists in that the dispersed droplets of the multiple emulsion encapsulate even smaller droplets of a phase similar (polyol) to the outer (water) phase. The composition obtained is a P/O/W (polyol-in-oil-in-water) multiple emulsion.

In short, the P/O/W emulsion is prepared in three steps: in the first step, the primary P/O (polyol-in-oil) emulsion is produced; in the second step the secondary O/W (oil-in-water) emulsion is produced; in the third step the P/O emulsion is dispersed in the O/W emulsion. One may further add actives such as vitamins or enzymes to the polyol phase.

This type of emulsion constitutes multiphase systems, which can enable multifunctionality. The inner phase may be prepared for encapsulating actives, including:
vitamins
enzymes
antiperspirant actives
fragrances
other components incompatible with the outer phase.

Further, the multiple-emulsion technology enables:
protection of the hydrophilic actives against oxidation and hydrolysis;
delay and control of the release of actives during a long period of time onto the skin; and
reduction of irritation by actives.

The multiple emulsion of the present invention is a multiphase system, intended to be used as an excipient for actives, acting as a “delivery system”.

In addition to the already cited components, it may also comprise stabilizing agents, network-forming agents, among other components known in the preparation of emulsions.

The multiple emulsion of the present invention has a number of advantages over the emulsions used in cosmetic compositions of the prior art, a few of them being listed below:
high efficacy with respect to the moisturizing of the skin, associated to properties such as pleasant smoothness, softness and texture; it combines the properties of moisturizing that is well-known of the oil emulsion (P/O) with easy spreadability and pleasant application of the aqueous emulsion (O/W) to the skin;
since the outer phase of the multiple emulsion is aqueous, there is an effect of immediate moisturizing when it is applied to the skin, a refreshing feeling and prolonged moisturizing;
encapsulation of actives such as vitamins and enzymes;
obtainment of controlled-release systems for actives, that is to say, an active dispersed in the inner droplets may be gradually released, thus promoting a prolonged effect;
the prolonged release of actives reduces irritation caused by determined actives, such as, for example, vitamin C;
association of immediate-release actives (present in the outer phase) with prolonged-release actives (present in the inner droplets);
use of actives that are incompatible with each other, since they are kept separate by a liquid membrane;
protection of instable actives, as for example vitamin C and enzymes, since it prevents contact of these actives with the destabilizing agents, which may be air (oxygen that causes oxidation) or water itself present in the formulations (which can promote hydrolysis);
this method enables the use of an inner aqueous phase with a different composition of the outer aqueous phase;
high stability (2-year average stability).
   I—Oil Phase: polyol-in-Oil Emulsion:
   Polyol

In the present invention, a polyol is used. It is selected from propylene glycol, butylene glycol, polyalkylene glycol, glycerol and polyglycerol. Preferably, propylene glycol is added as the polyol in an amount ranging from about 30% to about 50%, by weight, based on the total amount of the composition of the oil phase P/O.

Oil

In the oil phase of the multiple emulsion of the present invention, an oil is used. It is selected from silicone oils, paraffin oils, triglycerides, fatty alcohols, ester oils. In a preferred embodiment, silicone oil is used in an amount ranging from about 5% to about 30%, by weight, based on the total amount of the composition of the oil phase P/O.

Lipophilic Emulsifying Agent

Preferably, as lipophilic emulsifying agents are used silicones such as copolyol dimethicone, dimethicone, cyclomethicone, esters such as propylene glycol esters, among others.

The amount of this lipophilic emulsifying agent should be kept preferably between 5% and 30%, by weight, in the emulsion system, based on the weight of the composition of the oil phase. In this case, the preferred amount is of 10%, by weight, based on the total weight of the composition of the oil phase.

Mixed emulsifying agents can also be used, as long as they form a gel network.

Lipophilic Co-Emulsifying Agent

The combination of emulsifying agent and co-emulsifying agent is necessary for the interfacial film of the multiple droplets to be thicker and more stable as time passes. In this way, the stability of the present emulsion lasts for a period of 2 years, without phase separation. Preferably, as co-emulsifying agents are used silicone alkyl copolymer (the alkyl radical aids in stabilizing the actives, if the latter are present in the composition of the multiple emulsion), the mixture of cetyl dimethicone copolyol and polyglyceryl-4 isostearate, triglycerol-4 isostearate, in addition to berrenyl alcohol.

In preferred embodiments, at least one co-emulsifying agent is added in an amount raging from 5% to 30%, by weight, based on the total weight of the composition of the oil phase.

Electrolyte

Preferably, sodium chloride or magnesium sulfate is added to the multiple emulsion of the present invention as electrolytes. They act in various ways when present in the composition described:
in promoting the balance of the osmotic pressure between the inner polyol phase and the outer aqueous phase;
in encapsulating the polyol, leaving it less available for the hydrophilic portion of the emulsifying agent, allowing the emulsifying agent to be more lipophilic and capable of stabilizing emulsions of the O/W and PO type; and
providing a “salting out” effect, which limits the solubility of the emulsifying agents in the aqueous phase and concentrate them in the interface, forming a condensed and resistant film.

Preferably, the amounts range from 0.2% to 0.7%, which are added to the oil phase.

Network-Forming Agent

The film-forming agent is an optional constituent and acts in forming a network around the droplets and further provides the maintenance of the phase composed by polyol and, optionally, an active, as for example, ascorbic acid, inside the multiple droplet. By preference, polyvinylpyrrolidone is used as a network forming agent in an amount ranging from 0.2% to 3.0%, by weight, based on the total weight of the composition of the aqueous phase.

II—Aqueous Phase: Oil-in-Water Emulsion

Oil

The oil phase P/O is compatible with all the hydrophilic emulsifying agents that have more than 16 carbon atoms in their lipophilic hydrocarbon chain. Preferably, one uses, in the aqueous phase of the multiple emulsion of the present invention, an oil selected from silicone oils, paraffin oils, triglycerides, fatty alcohols, ester oils, propylene glycol and vegetable oils. In a preferred embodiment, silicone oil is used in an amount ranging from about 5% to about 30%, by weight, based on the total weight of the composition of the aqueous phase O/W.

Hydrophilic Emulsifying Agent

By preference, one uses, as hydrophilic emulsifying agents, Steareth 100, esters such as glycol esters, polyglycerol esters, sorbitan esters, sorbitol esters, fatty alcohols, among others.

The amount of this hydrophilic emulsifying agent should be kept preferably between 0.1% and 0.7%, by weight, based on the total weight of the composition of the aqueous phase.

One may also use mixed emulsifying agents, as long as they form a gel network. The amount of hydrophilic emulsifying agent should still be maintained below 0.7%.

Hydrophilic Co-Emulsifying Agent

The combination of emulsifying agents and co-emulsifying agents is necessary for the interfacial film of the multiple droplets to be thicker and more stable as times passes. Thus, the stability of the present emulsion lasts for a period of 2 years, without phase separation. Preferably, one uses, as co-emulsifying agents silicone alkyl copolymer (the alkyl radical aids in stabilizing the actives, if the latter are present in the composition of the multiple emulsion), the mixture of cetyl dimethicone copolyol and polygliceryl-4 isostearate as well as berrenyl alcohol.

In preferred embodiments, at least one co-emulsifying agent is added in an amount ranging from 5% to 30%, by weight, based on the total weight of the composition of the aqueous phase.

Thickening Agent

Optionally, a thickening agent may be added to the composition of the aqueous phase of the multiple emulsion so as to alter its viscosity. The primary emulsion P/O is compatible with virtually all the types of thickening agents. However, the best results are obtained with xanthan gum or the combination of xanthan gum and Pemulen, these thickening agents being preferred to be added in the present invention. The combination of xanthan gum and a texturing agent such as Dry Flo (aluminum octenyl succinate starch) imparts excellent stability and provides optimum feeling to the skin. Other examples of thickening agents indicated for the present invention are acrylates, C₁₀₋₃₀alkyl acrylate crospolymer and glyceryl monostearate.

This agent is also responsible for the stabilization of the multiple emulsion, since the stability of emulsions is directed related with the viscosity and inversely proportional to the particle size. Since the droplets of the present invention present in the multiple emulsion of the present invention are big (diameter of about 20 microns), the increase in viscosity of the outer phase being about stabilization of the emulsion.

The control of viscosity of the multiple emulsion is fundamental. If the size of the multiple particles is too small, the water droplets in the inner phase will be exposed to high pressure and may coalesce. On the other hand, if the particles are relatively large, they favor “creaming”.

In order to prevent “creaming”, the emulsion should have a viscoelastic behavior. This property is achieved by adding hydrocolloids such as xanthan gum or cellulose derivatives. Hydrocolloids also prevent creaming formation, which may occur due to the difference ion density between the aqueous phase and the oil phase.

Preferably, a thickening agent is added to the aqueous phase in an amount ranging from 0.1% to 1.0%, by weight, based on the total weight of the composition of the aqueous phase.

In addition to the components mentioned above, the multiple emulsion of the invention may further comprise, optionally, other components that are conventionally used in cosmetic compositions, which provide other characteristics that are not achieved by using the already described components:
emollient such as isohexadecane (heptamethylnonane), stearoxy dimethicone, hydrogenated polyisobutene, octyl salicilate, palm oil;
sunscreen such as butyl methoxydibenzoylmethane, octyl salicilate, Parsol 1789;
moisturizing agent such as white glycerin;
sequestering agent such as disodium EDTA;
texturing agent such as aluminum octyl succinate starch (Dry Fio);
preserving agent as phenoxyethanol, methyl paraben;
actives such as Camu-camu extract (it contains 30% of vitamin C), wine palm oil (contains β-carotene), ascorbic acid (vitamin C—is an anti-sign active and acts via stimulus of collagen synthesis and antiradical action), retinol (vitamin A—a cell renewing active), oily vitamin E, OPC, elastinol, proteins, glucose, among others.
   Release of Actives

The most important use of the multiple emulsion of the present invention refers to the release of actives, chiefly those listed above. Said release of actives may occur in two ways:
1—by coalescence of inner droplets, causing breakage of the oil droplet (multiple droplet), which are then released to the outer phase and/or
2—by diffusion through the oil phase (liquid membrane). The oil droplets act as a semi permeable membrane between the aqueous phase and the oil phase. The diffusion of the solute to the aqueous phase depends upon characteristics such as affinity with the oil phase, its dissociation constant, the pH of the phases, among others. An osmotic gradient may be created between the aqueous phase and the oil phase by using different concentrations of electrolytes, or with water-soluble actives such as proteins, glucose, glycerol, preserving agents, among others. The osmotic pressure increases the permeability of the oily liquid membrane, facilitating the transport of the oil phase to the aqueous phase.

The release of the active occurs slowly. Therefore, the multiple emulsion of the present invention enables a prolonged action of said actives on the substrate where it has been applied, preferably the skin.

Process of Preparing the Multiple Emulsion

There are a number of techniques that may be used to prepare the multiple emulsion of the present invention. The most recommended method is presented hereinafter.

In making the multiple emulsion, the primary emulsion P/O is dispersed, under controlled conditions and with addition of hydrophilic emulsifying agents, preferably of polymeric nature, to secondary emulsion O/W. The high steric hindrance supplied by polymers having a high molecular weight prevents the coalescence of the dispersed P/O emulsion.

The stirring velocity is very important at this stage. In general, low stirring is required for dispersing the primary emulsion in the secondary emulsion. Very intense stirring or homogenization induces the release of polyol droplets when the primary and secondary emulsions are already mixed.

The emulsion prepared according to the steps below comprises, in addition to the aqueous phase and oil phase, other components such as actives. This example should be understood as being illustrative, the addition of actives, thickening agents, network forming agents, moisturizing agents, emollient, sunscreen, texturing agent, sequestering agent being optional. Process of preparing the P/O phase
1—in a reactor, adding a polyol and an electrolyte, stirring and heating up to a temperature ranging from 80 to 85° C.;
2—adding at least one active and stirring for about 10 minutes;
3—after reaching total dissolution of the active, initiating the cooling to a temperature ranging from 30 to 26° C.;
4—adding a second active and stirring until total dissolution is reached;
5—in another reactor, adding at least one lipophilic emulsifying agent and at least one lipophilic co-emulsifying agent and then adding the previous mixture slowly with high stirring.

It is necessary to make a high stirring with shearing in order to produce a P/O emulsion with a small particle size.

Process of Preparing the P/O/W Emulsion

1—adding water, oil, hydrophilic emulsifying agent, moisturizing agent and sequestering agent in the main reactor, produce high stirring under vacuum and then adding a thickening agent. Mixing for about 7 minutes until total dispersion is reached;
2—heating up to a temperature of 75° C.;
3—adding emollients, sunscreens, hydrophilic emulsifying agents, hydrophilic co-emulsifying agents, actives in an auxiliary reactor, and heating until the temperature of 75° C. is reached;
4—Mixing for about 10 minutes with high stirring;
5—cooling down to a temperature ranging from 30 to 26° C.;
6—adding a second texturing agent and stirring for about 10 minutes;
7—adding the primary P/O emulsion under vacuum and middle stirring;
8—adding at least one preserving agent and other additives and mixing for 2 minutes.

In order to ensure that the active will not be diffused to the aqueous phase, intensive homogenization should be avoided after adding the primary P/O emulsion. It is recommended to use a naval-type propeller. To control the ideal distribution of the primary P/O emulsion in the emulsion, the particle size of the multiple droplets has to be controlled under a microscope. The ideal distribution of the particle size will be in the range from 5 to 20 μm.

It is further recommended to protect the emulsions containing ascorbic acid from the air during its manufacture, as well as filling up the empty space with nitrogen throughout the emulsifying process, since this prevents diffusion of micronized air within the primary P/O emulsion.

Actuation of the Multiple Emulsion in Application

The multiple emulsion enables a prolonged release of the actives onto the skin, that is to say, it allows said active to act longer on the skin, increasing its efficacy and also the tolerance of the skin to the product.

This prolonged effect is due to the large size of the multiple droplets. For this reason, they remain on the epidermis, permitting longer and more effective contact of the active.

Further, oxidation-sensitive actives such as vitamin C become more stable in the presence of propylene glycol and oil. This is due to the fact that the active remains involved by an oil membrane, which separates it from the outer aqueous phase and does not permit contact with air, thus preventing it from oxidizing.

### EXAMPLES OF COMPOSITION

Preferred embodiments having been described, it should be understood that the scope of the present invention embraces other possible variations, being limited only by the contents of the accompanying claims, which include the possible equivalents.

### Example 1

### P/O Oil Phase

1—in a reactor propylene glycol and magnesium sulfate are added, stirring and heating up to a temperature ranging from 80 to 85° C.;

2—adding ascorbic acid and retinol and stirring for about 10 minutes;

3—after reaching total dissolution of the actives, initiating the cooling down to a temperature ranging from 30 to 26° C.;

4—adding OPC and stirring until total dissolution is reached;

5—in another reactor, adding cetyl dimethicone copolyol, silsoft 034 and triglycerol-4 isostearate and then adding the previous mixture slowly under high stirring.

| Component | Mass amount (%) |
|---|---|
| Propylene glycol | 39.70 |
| Magnesium sulfate | 0.20 |
| Polyvinylpyrrolidone | 1.25 |
| Ascorbic acid | 1.00 |
| OPC | 0.05 |
| Retinol | 0.04 |
| Triglycerol-4 isostearate | 10.00 |
| Cetyl dimethicone copolyol | 10.00 |
| Silsoft 034 | 10.35 |

O/W Oil/Phase

1—adding water, propylene glycol, white glycerin and disodium EDTA in the main reactor, stirring under vacuum and then adding xanthan gum. Mixing for about 7 minutes or until total dispersion is achieved;

2—heating up to the temperature of 75° C.;

3—in an auxiliary reactor, adding steareth 100, berrenyl alcohol, glyceryl monostearate, heptamethylnonate, hydrogenated polyisobutene, dimethicone stearoxy, octyl salicinate, butyl methoxydibenzoylmethane and palm oil and heating up to a temperature of 75° C.;

4—mixing for about 10 minutes under high stirring;

5—cooling down to a temperature ranging from 30 to 26° C.;

6—adding aluminum octenyl succinate starch and stirring for about 10 minutes.

P/O/W Multiple Emulsion

1—adding the primary P/O emulsion in the secondary O/W emulsion under vacuum and middle stirring;

2—adding phenoxyethanol and methyl paraben and mixing for 2 minutes.

| Component | Mass amount (%) |
|---|---|
| Steareth 100 | 0.20 |
| Berrenyl alcohol | 1.50 |
| Glyceryl monostearate | 1.20 |
| heptamethylnonane | 9.00 |
| Dimethicone stearoxy | 2.70 |
| Hydrogenated polyisobutene | 4.00 |
| Octyl salicilate | 2.00 |
| Butyl methoxydibenzoylmethane | 0.50 |
| Palm oil | 0.50 |
| Water | 58.30 |
| White glycerin | 2.70 |
| Disodium EDTA | 0.20 |
| Xanthan gum | 0.20 |
| Propylene glycol | 5.00 |
| Aluminum octenyl succinate starch | 1.50 |
| Phenoxyethanol, methyl paraben | 0.50 |
| P/O oil phase | 10.00 |

| Component | Mass amount (%) |
|---|---|
| Propylene glycol | 39.70 |
| Sodium chloride | 0.20 |
| Polyvinylpyrrolidone | 1.25 |
| Camu-camu extract | 22.50 |
| Wine-palm oil | 5.00 |
| Alpha-tocopherol | 1.00 |
| Triglycerol-4 isostearate | 10.00 |
| Cetyl dimethicone copolyol | 10.00 |
| Silsoft 034 | 10.35 |

| Component | Mass amount (%) |
|---|---|
| Steareth 100 | 0.50 |
| Berrenyl alcohol | 1.50 |
| Glyceryl stearate | 1.20 |
| Heptamethylnonane | 8.00 |
| Oily vitamin E | 1.00 |
| Cetyol OE | 7.50 |
| Hydrogenated polyisobutene | 1.00 |
| Water | 51.94 |
| White glycerin | 3.00 |
| Dequest | 0.15 |
| TR1 alkyl acrylate | 0.30 |
| Xanthan gum | 0.25 |
| Propylene glycol | 5.00 |
| Aluminum octenylsuccinate starch | 1.50 |
| Fucogel 1000 | 3.00 |
| Glycacil L | 0.20 |
| Phenoxyethanol F | 0.70 |
| Wine-palm essence | 0.10 |
| Water | 3.00 |
| Triethanolamine | 0.16 |
| P/O oil phase | 10.00 |

| Component | Mass amount (%) |
|---|---|
| Propylene glycol | 39.70 |
| Sodium chloride | 0.20 |
| polyvinylpyrrolidone | 1.25 |
| Camu-camu extract | 22.50 |
| Wine-palm oil | 5.00 |
| Alpha-tocopherol | 1.00 |
| Triglycerol-4 isostearate | 10.00 |
| Cetyl dimethicone copolyol | 10.00 |
| Cyclomethicone | 10.35 |

| Component | Mass amount (%) |
|---|---|
| Steareth 100 | 0.50 |
| Berrenyl alcohol | 1.50 |
| Glyceryl stearate | 1.20 |
| Heptamethylnonane | 8.00 |
| Oily vitamin E | 1.00 |
| Cetiol OE | 7.50 |
| Hydrogenated polyisobutene | 1.00 |
| Water | 52.00 |
| White glycerin | 3.00 |
| Dequest | 0.15 |
| TR1 alkyl acrylate | 0.25 |
| Xanthan gum | 0.25 |
| Propylene glycol | 5.00 |
| Aluminum octenylsuccinate starch | 1.50 |
| Fucogel 1000 | 3.00 |
| Glycacyl L | 0.20 |
| Phenoxyethanol F | 0.70 |
| Wine-palm essence | 0.14 |
| Water | 3.00 |
| Triethanolamine | 0.21 |
| Polyol-in-oil with vitamin A, C and E | 10.00 |

| Component | Mass amount (%) |
|---|---|
| Propylene glycol | 38.45 |
| Sodium chloride | 0.20 |
| Polyvinylpyrrolidone | 2.50 |
| Camu-camu extract | 22.50 |
| Wine-palm oil | 5.00 |
| Alpha-tocopherol | 1.00 |
| Triglycerol-4 isostearate | 10.00 |
| Cetyl dimethicone copolyol | 10.00 |
| Cyclomethicone | 10.35 |

| Component | Mass amount (%) |
|---|---|
| Steareth 100 | 0.60 |
| Berrenyl alcohol | 1.50 |
| Glyceryl stearate | 1.20 |
| heptamethylnonane | 6.00 |
| Oily vitamin E | 1.00 |
| Cetiol OE | 5.50 |
| Water | 56.67 |
| White glycerin | 3.00 |
| Dequest | 0.15 |
| TR1 alkyl acrylate | 0.30 |
| Xanthan gum | 0.25 |
| Propylene glycol | 5.00 |
| Aluminum octenylsuccinate starch | 1.50 |
| Fucogel 1000 | 3.00 |
| Glycacil L | 0.20 |
| Phenoxiethanol F | 0.70 |
| Wine-palm essence | 0.10 |
| Water | 3.00 |
| Triethanolamine | 0.28 |
| P/O oil phase | 10.00 |

| Component | Mass amount (%) |
|---|---|
| Propylene glycol | 49.7 |
| Sodium chloride | 0.20 |
| OPC | 0.5 |
| Ascorbic acid | 10.0 |
| Retinol, polysorbate | 0.4 |
| Cyclomethicone copolyol and | 20.2 |
| dimethicone | |
| Polyvinylpyrrolidone | 2.50 |
| Cetyl dimethicone copolyol | 9.5 |
| Cyclomethicone | 9.5 |

| Component | Mass amount (%) |
|---|---|
| Steareth 100 | 0.30 |
| Berrenyl alcohol | 1.50 |
| Glyceryl stearate | 1.20 |
| Heptamethylnonane | 8.00 |
| Dimethicone | 1.00 |
| Polyisobutene | 4.00 |
| Octyl salicilate | 2.00 |
| Butyl methoxydibenzoylmethane | 0.50 |
| (Parsol 1789) | |
| Palm oil | 0.50 |
| Cyclomethicone | 1.70 |
| Oil vitamin E | 1.00 |
| Water | 56.00 |
| White glycerin | 2.70 |
| Disodium EDTA | 0.20 |
| Propylene glycol | 5.00 |
| Xanthan gum | 0.40 |

Tests

Below, brief explanations are given on the tests carried out to prove the already disclosed properties of the multiple emulsion of the present invention. The composition of the multiple emulsion used in all the tests is that described in example 5 of the present specification, indicated as Product 1 in the information given hereinafter.

Firsts Test: Analysis of Cutaneous Permeation

One has made studies on in vitro permeation on animal (hairless mouse) and studies on in vitro cutaneous retention, in the stratum corneum and in the epidermis/dermis assembly. With this latter study, one determines the amount of vitamin C that is retained in the horny layers. Two multiple emulsions according to the invention were used, which contain L-ascorbic acid and a pattern of L-ascorbic acid from Merck, the reference of which is 5,00074 H564374.

The results achieved in the cutaneous permeation tests show that the multiple emulsions studied present lower cutaneous permeation than when compared with the referred-to pattern, but more constant. It is important to point out that the term cutaneous permeation refers to the penetration of actives as far as the hypodermis or blood circulation. The ideal performance of a cosmetic product of topical application is a high release in the superficial layers of the skin (local effect) and a low permeation (systemic effect).

Result: the multiple emulsion exhibits a release profile more suitable for vitamin C when compared with the standard. Further, the multiple emulsion of the present invention enables one to maintain the skin in contact with vitamin C for a longer period of time, that is to say, promoting prolonged action.

Second Test: Analysis of the Dosage of Vitamin C

In this test one has studied a multiple emulsion (product 1) and a cosmetic composition that basically comprises a simple emulsion of vitamin C (product 2).

It has been found that the multiple emulsion enables stabilization of vitamin C in the inner phase (polyol). After 90 days from the preparation of each of the products, product 1 exhibited stability of 84% of the vitamin C contained therein. On the other hand, product 2 exhibited 56% of stable vitamin C. Therefore, product 1 comprises 33% more vitamin C after the 90-day period.

Result: in all the above conditions, vitamin C remained more stable inserted in the multiple emulsion of the present invention.

Third Test: Evaluation of the Performance and Preference of Users

For sensorial evaluation of the Performance and Preference of Users, one used the GAP methodology (quantitative internal study, used a questionnaire filled up by 48 volunteers about several characteristics of the product containing the multiple emulsion of the present invention).

The performance of the multiple emulsion (Product 1) has compared with that of a product containing a simple emulsion (Product 2).

The result of this test indicated the general preference of 56.3% of the volunteers for Product 1 versus 43.8% of the volunteers for Product 2. Further, 81.3% of the volunteers classified Product 1 as being good and very good versus 70.8% for Product 2.

Result: the multiple emulsion enables one to add chemical filters without loss of touch and smoothness of the emulsion.

Fourth Test: Evaluation of Toxicology

The toxicological tests carried out showed that the multiple emulsion tested is not irritant. Below, one indicates the simplified methodology used in each of the toxicology tests.
Evaluation of ocular irritation: 5 albino rabbits were clinically examined. The volume of 0.1 ml of sample of Product 1 was placed in one of the rabbit's eyes and the other eye served as control. The ocular reactions were measured at definite intervals of time.

Result: the tests for ocular irritation carried out on albino rabbits have shown that the multiple emulsion is not irritant.
Evaluation of primary dermal irritation: 6 albino rabbits were clinically used. The sites for analysis were determined on the shaved skin of the animals, and Product 1 was applied to them. The dermal reactions were measured at definite intervals of time.

Result: the tests for primary dermal irritation carried out on albino rabbits have shown that the multiple emulsion is not irritant.
Evaluation of cumulative dermal irritation: 6 albino rabbits were clinically examined. The sites for analysis were determined on the shaved skin of the animals. The product was applied to the sites for 10 consecutive days. The dermal reactions were measured at definite intervals of time.

Result: the tests for cumulative dermal irritation carried out on albino rabbits have shown that the multiple emulsion is not irritant.
Evaluation of dermal photoirritation: 6 albino rabbits were clinically examined. The sites for analysis were determined. The sites for analyses were determined on the shaved back region of the animals. The product was applied to the sites, which then underwent irradiation of a solar simulator. The dermal reactions were measured at definite intervals of time.

Result: the tests for dermal photoirritation carried out on albino rabbits have shown that the multiple emulsion does not have any photoirritant potential.

Fifth Test: Evaluation of Stability

The stability of the multiple emulsion was tested under these conditions: dark, light, 5° C. and 45° C., for 3 months. The product was stable in the first 3 conditions. At a temperature of 45° C., after a period of 30 days, there was separation of the phases, which does not impair the validity term of the product.

1. A multiple emulsion of the P/O/W type, which is constituted by two phases: an aqueous phase composed of an oil-in-water emulsion and an oil phase composed of a polyol-in-oil emulsion, characterized in that said oil phase comprises at least one lipophilic emulsifying agent, at least one lipophilic co-emulsifying agent and at least one electrolyte.

2. A multiple emulsion according to claim 1, characterized in that the amount of polyol contained in the oil phase ranges from 30% to 50%, by weight, based on the total weight of the composition.

3. A multiple emulsion according to claim 1, characterized in that the polyol is selected from propylene glycol, butylene glycol, polyalkylene glycol, glycerol and polyglycerol.

4. A multiple emulsion according to claim 3, characterized in that the polyol is propylene glycol.

5. A multiple emulsion according to claim 1, characterized in that the oil phase comprises an amount of oil ranging from 5% to 30%, by weight, based on the total weight of the composition.

6. A multiple emulsion according to claim 1, characterized in that the oil is selected from silicone oils, paraffin oils, triglycerides, fatty alcohols and ester oils.

7. A multiple emulsion according to claim 6, characterized in that the oil is silicone oil.

8. A multiple emulsion according to claim 1, characterized in that the amount of lipophilic emulsifying agent present in the oil phase ranges from 5% to 30%, by weight, based on the total weight of the composition of the oil phase.

9. A multiple emulsion according to claim 1, characterized in that the lipophilic emulsifying agent is selected from copolyol dimethicone, dimethicone, cyclomethicone, cetyl dimethicone, esters such as polyglycerol esters and mixtures thereof.

10. A multiple emulsion according to claim 9, characterized in that the emulsifying agent is cetyl dimethicone.

11. A multiple emulsion according to claim 1, characterized in that the amount of lipophilic co-emulsifying agent ranges from 5% to 30%, by weight, based on the amount of the P/O oil phase.

12. A multiple emulsion according to claim 1, characterized in that the co-emulsifying agent is selected from silicone copolymer alkyls, berrenyl alcohol, and a mixture of cetyl dimethicone copolyol and polyglyceril-4 isostearate.

13. A multiple emulsion according to claim 12, characterized in that the co-emulsifying agent is polyglyceryl-4 isostearate.

14. A multiple emulsion according to claim 1, characterized in that the amount of electrolyte ranges from 0.2% to 0.7%, by weight, based on the amount of the composition of the aqueous phase.

15. A multiple emulsion according to claim 1, characterized in that the electrolyte is selected from sodium chloride and magnesium sulfate.

16. A multiple emulsion according to claim 1, characterized in that at least one active is added to the oil phase.

17. A multiple emulsion according to claim 16, characterized in that the active is selected from Camu-camu extract, wine palm oil, ascorbic acid, retinol, oily vitamin E, OPC, elastinol, proteins, glucose, enzyme and combinations thereof.

18. A multiple emulsion according to claim 1, characterized in that a network-forming agent is added to the oil phase in an amount ranging from 0.2% to 3.0% by weight, based on the amount of the composition of said oil phase.

19. A multiple emulsion according to claim 18, characterized in that the network-forming agent is polyvinylpyrrolidone.

20. A multiple emulsion according to claim 1, characterized in that a thickening agent is added in the aqueous phase in an amount ranging from 0.1% to 1.0%, by weight, based on the amount of the composition of said aqueous phase.

21. A multiple emulsion according to claim 20, characterized in that the thickening agent is selected from xanthan gum, Pemulen, acrylates, C₁₀₋₃₀alkyl acrylate crospolymer and glyceryl monostearate.

22. A multiple emulsion according to claim 1, characterized in that an emollient is added selected from isohexadecane, dimethicone stearoxy, hydrogenated polyisobutene, octyl salicilate, palm oil and combinations thereof.

23. A multiple emulsion according to claim 1, characterized in that at least one sunscreen is added selected from butyl methoxydibenzoylmethane, octyl salicilate, Parsol 1789.

24. A cosmetic product characterized by comprising the multiple emulsion defined in claim 1.
